(19) United States
(12) Patent Application Publication
(10) Pub. No.: US 2024/0148501 A1
(43) Pub. Date: May 9, 2024

(54) Prosthetic Heart Valve Delivery and Trackability

(71) Applicant: St. Jude Medical, Cardiology Division, Inc., St. Paul, MN (US)

(72) Inventors: Neil Theisen, Champlin, MN (US); Michael Shane Morrissey, St. Paul, MN (US); Tyler Govek, St. Louis Park, MN (US); Hannah Thatcher, St. Paul, MN (US); Hans Rieckmann, Minneapolis, MN (US); Robert Schulzetenberg, Columbia Heights, MN (US); Peter J. Ness, Minneapolis, MN (US); Victoria Schuman, Minneapolis, MN (US)

(73) Assignee: St. Jude Medical, Cardiology Division, Inc., St. Paul, MN (US)

(21) Appl. No.: 18/498,556

(22) Filed: Oct. 31, 2023

Related U.S. Application Data
(60) Provisional application No. 63/382,812, filed on Nov. 8, 2022.

(51) Int. Cl. A61F 2/24

(52) U.S. Cl. CPC A61F 2/2433; A61F 2/2418; A61F 2/2436

(57) ABSTRACT

A delivery device includes a catheter, and an inflatable balloon coupled to the catheter, the inflatable balloon forming a leading pillow and a trailing pillow spaced from the leading pillow, the leading pillow and the trailing pillow defining a valve seat therebetween to retain a prosthetic heart valve during tracking of the delivery device.

### CROSS-REFERENCE TO RELATED APPLICATIONS

This application claims priority from U.S. Provisional Application No. 63/382,812, filed Nov. 8, 2022, the contents of which is incorporated by reference in its entirety as if fully set forth herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Acceptable trackability of TAVR devices is desirable to ensure atraumatic contact with the vasculature, as well as reliable implant performance, which includes proper valve retention about the delivery device during the deliver process. This is especially true for prosthetic valves that are exposed to the anatomy during tracking such as most balloon expandable valves. Embodiments described herein may generally relate to features for improving trackability of a TAVR device and/or delivery device, and for improving TAVR device retention during delivery and deployment.

### BRIEF SUMMARY OF THE DISCLOSURE

A delivery device includes a catheter, and an inflatable balloon coupled to the catheter, the inflatable balloon forming a leading pillow and a trailing pillow spaced from the leading pillow, the leading pillow and the trailing pillow defining a valve seat therebetween to retain a prosthetic heart valve during tracking of the delivery device.

A method of delivering a prosthetic heart valve, the method including providing a delivery device having a catheter and an inflatable balloon coupled to the catheter, forming a leading pillow and a trailing pillow spaced from the leading pillow on the balloon, the leading pillow and the trailing pillow defining a valve seat, placing a prosthetic heart valve on the valve seat, and advancing the delivery device to a native aortic valve of a patient while the prosthetic heart valve is disposed between the leading pillow and the trailing pillow of the delivery device.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIGS. 3A-B are schematic illustrations of a balloon before and after forming pillows.

FIGS. 4A-C are schematic illustrations showing the formation of pillows on a balloon via a heat setting process.

FIGS. 5A-B are schematic illustrations of a balloon having shoulders.

FIGS. 6A-B are schematic illustrations of the foreshortening of a balloon during pillow formation.

FIG. 6C is a schematic illustration of the inclusion of curved cones to improve trackability.

FIGS. 7A-B are schematic illustrations of internal retention elements.

FIGS. 8A-D are schematic illustrations of several examples of balloons having pillows.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

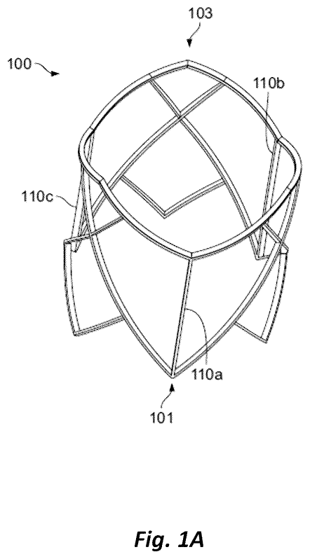

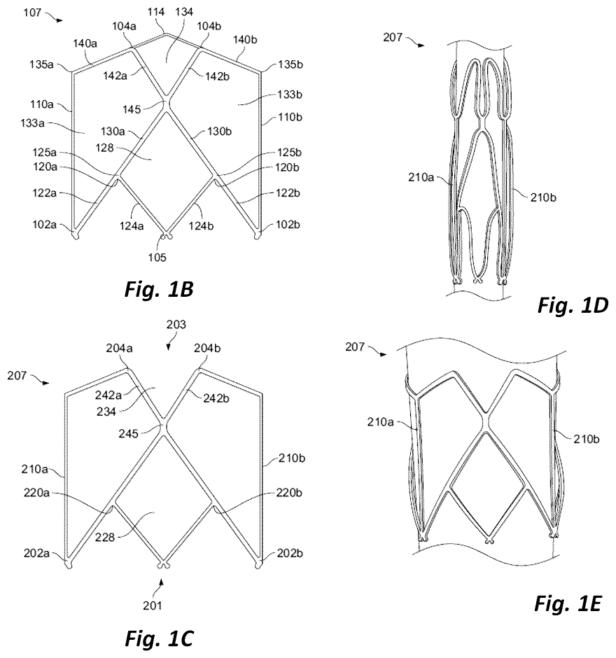

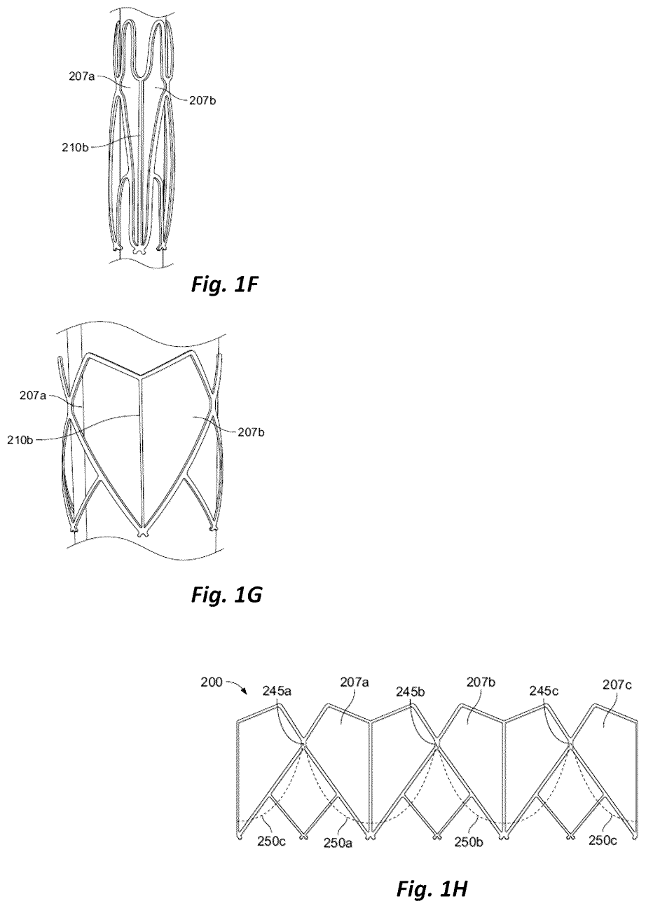

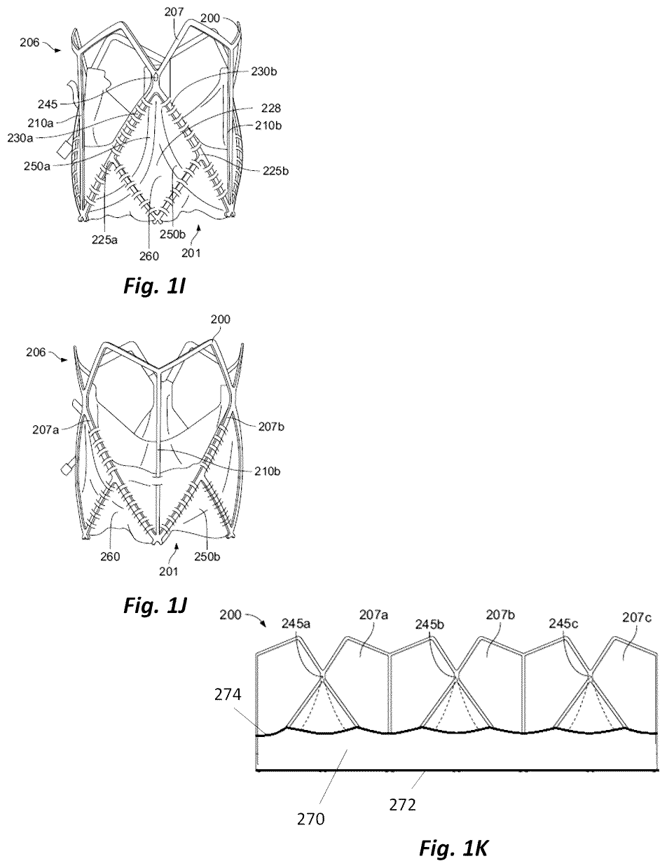

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

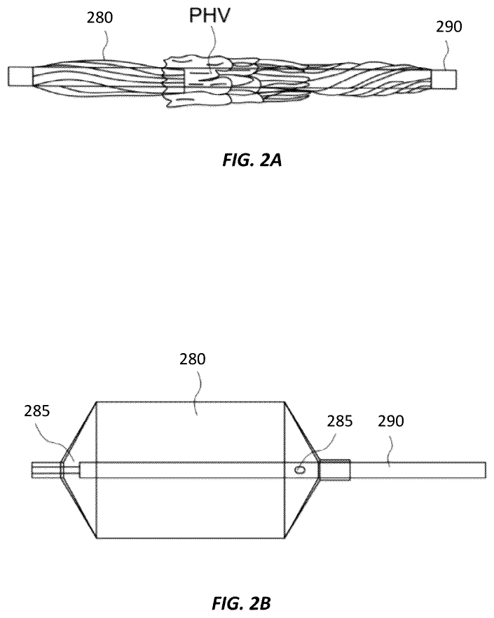

Referring to FIG. 2A, an example of a prosthetic heart valve PHV, which may include a stent similar to stents 100 or 200, is shown crimped over a balloon 280 of a balloon catheter 290 while the balloon 280 is in a deflated condition. It should be understood that other components of the delivery device, such as a handle used for steering and/or deployment, as well as a syringe for inflating the balloon 280, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 290 to inflate the balloon 280, as shown in FIG. 2B. FIG. 2B omits the prosthetic heart valve PHV, but it should be understood that, as the balloon 280 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 280 through a lumen within balloon catheter 290 and into one or more ports 285 located internal to the balloon 280. In the particular illustrated example of FIG. 2B, a first port 285 may be one or more apertures in a side wall of the balloon catheter 290, and a second port 285 may be the distal open end of the balloon catheter 290, which may terminate within the interior space of the balloon 280.

During normal operation of a prosthetic heart valve, the prosthetic leaflets open and close cyclically as the chambers of the heart contract and relax. For example, when the left ventricle relaxes and the left atrium contracts, the mitral valve opens and the aortic valve closes. For a prosthetic aortic valve, as the left ventricle relaxes, the prosthetic leaflets coapt to prevent blood from flowing in the retrograde direction from the aorta back into the left ventricle. As the prosthetic leaflets open and close, and particularly when they close, the prosthetic leaflets can encounter stress as the prosthetic leaflets resist the pressure gradient across the closed valve assembly. This stress may largely act at the point(s) where the prosthetic leaflets are affixed to the frame (or an intermediary component). Because prosthetic heart valves may need to last years, decades, or more, it may be important to minimize the amount of stress experienced by the prosthetic leaflets during normal operation to reduce the amount of wear and tear on the prosthetic leaflets, since such wear and tear may reduce the longevity of the prosthetic leaflets. One way to reduce stress on the prosthetic leaflets is to allow for deflection of the structure to which the prosthetic leaflets are attached. For example, if prosthetic leaflets are directly sutured to a commissure attachment feature of a frame, allowing the frame to deflect slightly (e.g., about 1 mm) as the prosthetic leaflets close may help reduce the stresses on the prosthetic leaflets as they coapt.

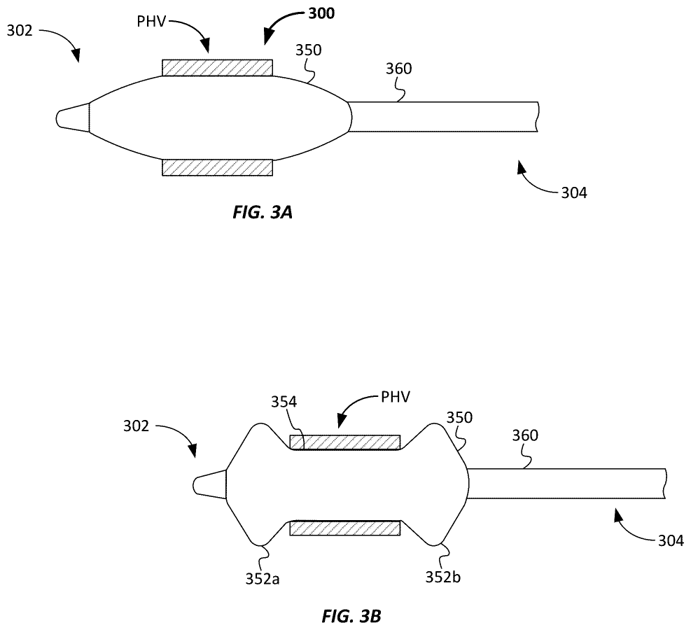

The present disclosure provides various embodiments to improve trackability, minimize transition along a delivery device length, and/or improve valve retention during delivery or deployment. It will be understood that that the embodiments described herein are illustrative and that the principles of the embodiments may be combined with one another. FIG. 3A illustrates a delivery system 300 that extends from a distal end 302 to a proximal end 304, the delivery system having a balloon 350 coupled to a delivery catheter 360, and a prosthetic heart valve PHV disposed about balloon 350. Placing prosthetic heart valve PHV radially outward of balloon 350 may present trackability and valve retention concerns as previously described. Instead, a balloon pillowing process may be performed on balloon 350 prior to the delivery and/or implantation. As shown in FIG. 3B, balloon 350 may be manufactured to include a leading pillow 352a, a trailing pillow 352b, and a substantially linear seat 354 extending between the two pillows 352a,352b and capable of receiving prosthetic heart valve PHV therein. The pillows 352a,352b may be formed in a variety of manners. In one example, the pillowing of balloon 350 occurs during the loading process and includes holding the prosthetic heart valve PHV at a predetermined constant diameter while partially inflating the balloon 350 to create the pillows shown in FIG. 3B. That is, pillows 352a,352b are not initially present, but are formed after prosthetic heart valve PHV is placed on the balloon, and the balloon transitions from a deflated condition to a partially-inflated loading condition. After loading, pillows 352a,352b may be present during the delivery device with prosthetic heart valve PHV securely placed therebetween. Alternatively, the pillows may be formed in a catheterization laboratory via a puffing process by injecting a small volume of inflation medium into the balloon to form gradual ramps or pillows as shown in FIG. 3B.

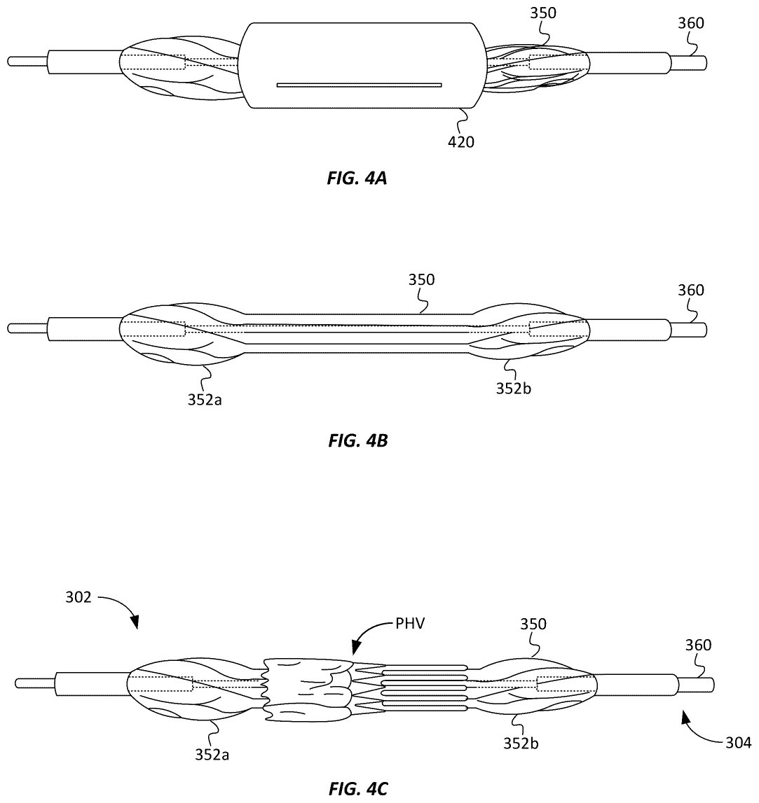

In another embodiment, the pillows may be pre-formed via heat setting and the pillowed balloon may be delivered to the operator ready for loading. FIGS. 4A-C illustrate various steps for forming the pillows without prosthetic heart valve PHV. In FIG. 4A, balloon 350 may be placed within a hollow cylindrical die 420, and the die may be used to heat set the balloon into a pre-pillowed state. It will be understood that the shape of the die 420 may result in variable shapes and curvatures to define one or more of a body diameter, a cone diameter and/or a shoulder geometry, or to customize and control the ramp from distal tip to implant. After the die 420 is removed, the pre-pillowed state is formed and the pillows 352a,352b are present before the prosthetic heart valve PHV loading process (FIG. 4B). A prosthetic heart valve PHV may then be loaded over balloon 350 between pillows 352a,352b for delivery (FIG. 4C).

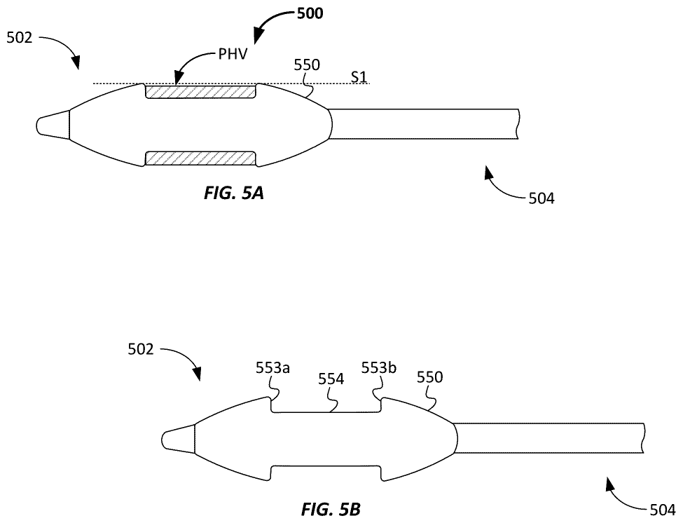

FIGS. 5A-B illustrate the inclusion of balloon shoulders. As shown, delivery system 500 extending between a distal end 502 and a proximal end 504 and includes a balloon 550 and a prosthetic heart valve PHV disposed about the balloon. In this example, balloon 550 includes a radially-extending leading shoulder 553a and a radially-extending trailing shoulder 553b that are orthogonal to seat 554, the seat 554 being of a sufficient length to hold the prosthetic heart valve PHV between the shoulders 553a,553b. The inclusion of shoulders 553a,553b may aid in holding the prosthetic heart valve PHV in placed during tracking and expansion, and may create an atraumatic transition between balloon 550 and prosthetic heart valve PHV to control and minimize any gaps therebetween. This may be desirable as edges of the stent crimped on a catheter without atraumatic features may cause trauma to the vasculature especially as the catheter is forced through curved vessels of small radii. In some examples, the shoulders have a predetermined height that is equal to or greater than a thickness of prosthetic heart valve PHV. Stated another way, when properly disposed within seat 554 of balloon 550, prosthetic heart valve PHV may be sunken below, or aligned with, shoulder line S1.

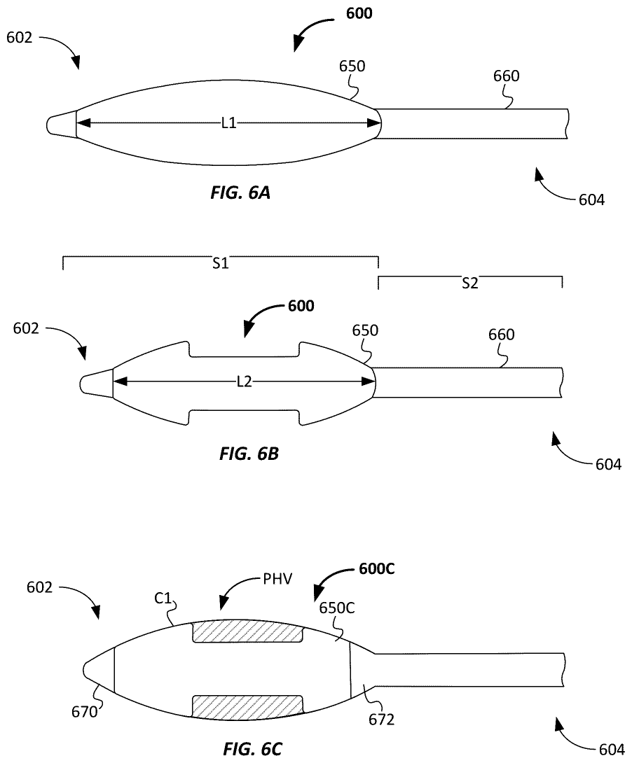

FIGS. 6A-B illustrate yet another embodiment of a delivery system 600 that extends between a distal end 602 and a proximal end 604. In this example, balloon 650 coupled to delivery catheter 660 may have a first balloon length L1 (FIG. 6A), and the balloon 650 may axially contract under partial inflation when the pillows, shoulders or trackability features are formed. As shown in FIG. 6B, balloon 650 has contracted to a second balloon length L2 that is less than first balloon length L1. The resulting delivery system may have improved trackability due to the shortened section S1, which is relatively stiff when compared to the proximal catheter shaft section S2. In some examples, a shorter balloon length may provide better steering of the valve when it is tracked around the aortic arch. This is because our intended point of steering is at the proximal end of the balloon where the steerable shaft ends. The shorter the balloon length, the more deflection of the valve is achievable. Additionally, the process of pushing more material into the center crimp section may aid in reducing second balloon length make L2. In some examples, the first length is between 60 and 66 mm (e.g., 65.6 mm) and the second length is between 50 and 65 mm (e.g., 62 mm). This may also reduce the foreshortening during balloon inflation.

In another variation, shown in FIG. 6C, a delivery system 600C may include curved cones 670,672 on either side of a balloon, and formed for introducing the balloon 650C into sheaths and navigating vasculature, insertion through stenotic native valve, insertion through existing bioprosthetic valves, withdrawing from vasculature and sheath to provide more seamless transitions and/or the ability to withdraw valve for a potential bailout. The curved cones may include a leading curved cone 670 and a trailing curved cone 672 and the two, along with the balloon, may define a continuous curvature C1 that extends from the leading curved cone 670 through the balloon 650C to the trailing curved cone. In at least some examples, the two cones and the balloon 650 may form an oblate spheroid shape, an egg-shaped or a football shape.

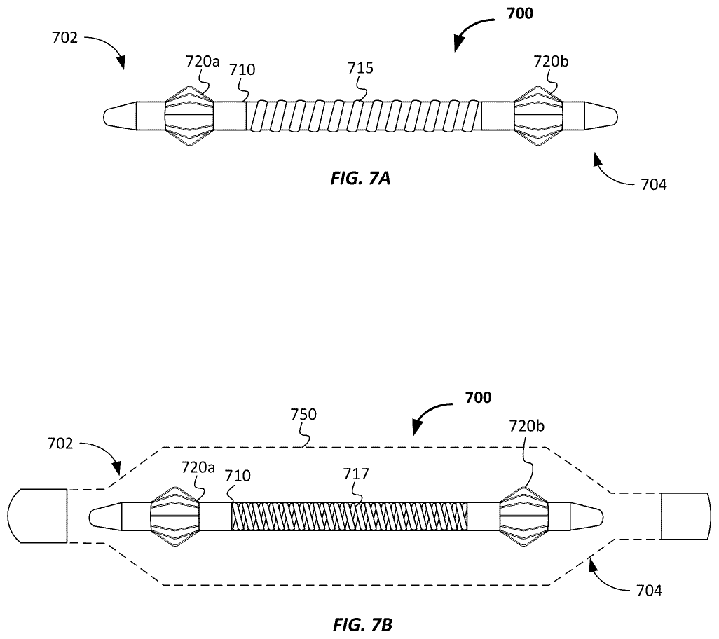

In at least some examples, additional internal features may be used in conjunction with the pillows described above to aid in trackability. For example, as shown in FIG. 7A, delivery system 700 may extend between distal end 702 and proximal end 704 and include an inner shaft 710 comprising a helical tube 715. In at least some examples, helical tube 715 is nitinol, and all or portions of inner shaft 710 may be formed of the helical tube. Expanded or expandable cages 720a,720b may be disposed on opposing ends of inner shaft 710. In at least some examples, cages 720a,720b are formed of an expandable nitinol basket. The inner shaft, helical tube and cages may collectively form internal valve retentions features configured to be disposed within the balloon, and the prosthetic heart valve PHV may be crimped onto, or between, the internal retention features. FIG. 7B shows a similar delivery system except that helical tube 715 has been replaced with a braided wire tube 717 comprising, for example, nitinol, and the whole assembly is shown within balloon 750.

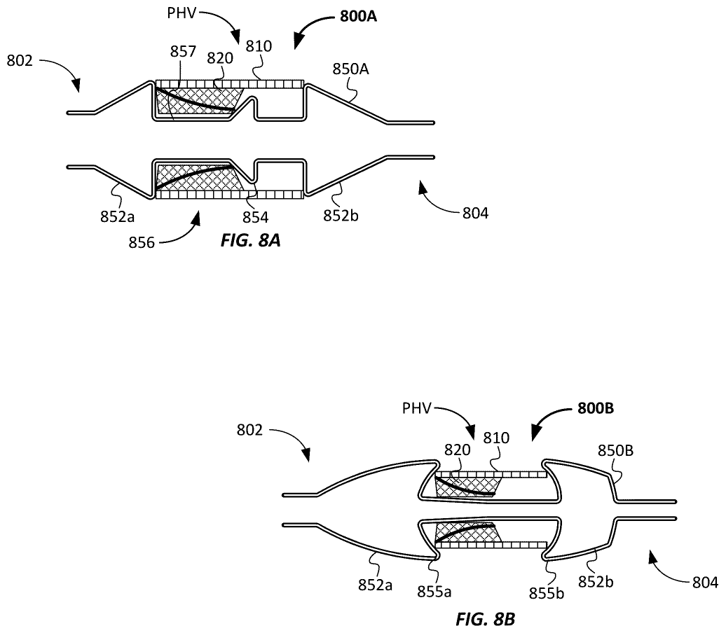

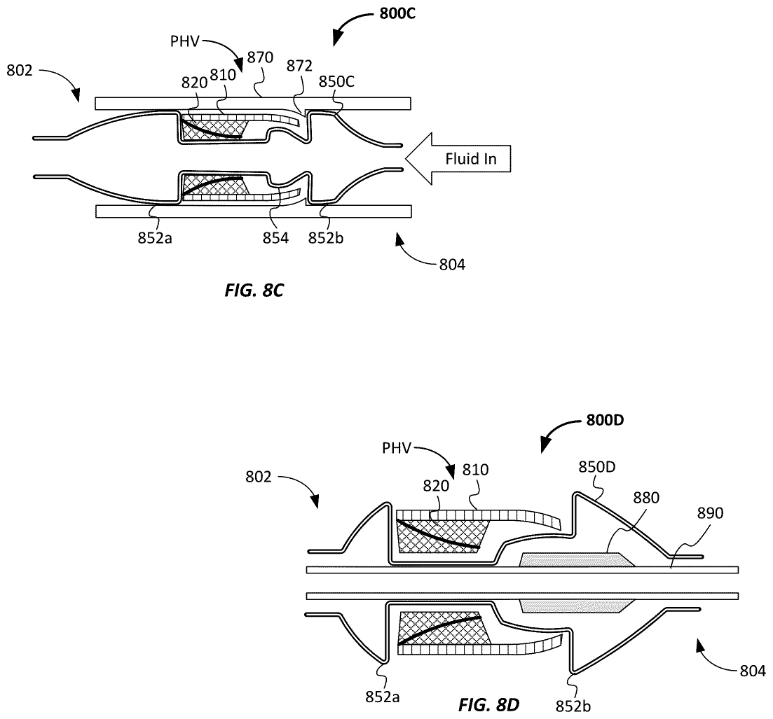

The embodiments above generally describe balloons with two-pillow configurations, but it will be understood that a single pillow or that three or more pillows may be used improve trackability. In one example, shown in FIG. 8A, a delivery system 800A may extend between distal end 802 and proximal end 804 and include a balloon 850A having a leading pillow 852a and a trailing pillow 852b. Balloon 850 may also have an intermediate pillow 854 disposed between the leading and trailing pillows 852a,852b. As shown, when a prosthetic heart valve PHV is disposed about the balloon, a stent 810 may be disposed between the leading and trailing pillows 852a,852b. The bulk of the valve assembly 820 of prosthetic heart valve PHV including, for example, the leaflets, one or more cuffs, paravalvular leakage components, and/or anchoring components may be disposed between leading pillow 852a and intermediate pillow 854 within a valve cavity 856. A majority or an entirety of the valve assembly maybe disposed about first seat 857 between pillow 852a and intermediate pillow 854, but it will be understood that other portions of prosthetic heart valve PHV (e.g., a portion of the cuff near an aortic end of the stent) may extend past intermediate pillow 854. Intermediate pillow 854 may be equidistant to the leading pillow and the trailing pillow or may be closer to one than the other. Intermediate pillow 854 may vary in shape and/or size. In some examples, intermediate pillow 854 is in the shape of a “mini-pillow” that is shorter in a radial direction and/or narrower in an axial direction than leading and/or trailing pillows 852a,852b.

In another example, shown in FIG. 8B, a delivery system 800B may extend between distal end 802 and proximal end 804 and include a balloon 850B having a leading pillow 852a and a trailing pillow 852b. Balloon 850B may be similar to those recited above and formed in any of the ways previously described. As shown, when a prosthetic heart valve PHV is disposed about the balloon, a stent 810 and valve assembly 820 may be disposed between the leading and trailing pillows 852a,852b. In this example, pillows 852a,852b are formed to have overhanging lips 855a,855b, respectively that at least partially extend over stent 810 to secure prosthetic heart valve PHV. In at least some examples, overhanging lips 855a,855b are configured to cover the leading and/or trailing end of stent 810 to reduce the edges catching on the anatomy or other environmental structures during delivery.

In FIG. 8C, the use of a loader sheath is shown to ensure that pillows retain atraumatic edges during preparation/de-airing of a delivery system in the catheterization laboratory. A delivery system 800C may extend between distal end 802 and proximal end 804 and include a balloon 850C having a leading pillow 852a, a trailing pillow 852b and an intermediate pillow 854 to secure the valve bulk 820. In this example, a loader sheath 870 is disposed over the balloon 850C, the loader sheath 870 having an optional radially inward protruding ramp 872 to retain stent 810 close to balloon 850C after the deairing process. Specifically, during deairing of the balloon 850C, fluid is injected, which pressurizes the balloon. Without protruding ramp 872, the stent and specifically the struts at the end of the stent may expand to a diameter larger than trailing pillow 852b. In this example, the leading pillow 852a may also act as a stopper to prevent the valve from shifting proximally during insertion and traversing the anatomy. Also notable in this example is that stent 810 may be asymmetrically crimped (e.g., it may be crimped to a first diameter near distal end 802 and a second diameter, smaller then the first diameter, near proximal end 804 or vice versa). In some examples, the proximal end of the stent may be crimped to between 4 mm and 6 mm, and the distal end of the stent may be crimped to between 6 mm and 8 mm. In some examples, the difference between the proximal end crimped diameter and the distal end crimped diameter is between 1 mm and 3 mm, the proximal end being smaller. In some examples, the stent may be crimped so that the crimped diameter of the stent has a proximal end-to-distal end crimped diameter ratio of between 60% and 90%.

In another example, shown in FIG. 8D, kink prevention features are described that reinforce a balloon inner shaft (otherwise referred to as a balloon inner catheter or BIC) in areas vulnerable to kinking during tracking. A delivery system 800D may extend between distal end 802 and proximal end 804 and include a balloon 850D having a leading pillow 852a and a trailing pillow 852b to secure prosthetic heart valve PHV including stent 810 and valve assembly 820. In this example, rigid kink prevention features 880 may be disposed about an inner shaft 890 that extends through balloon 850D. Kink prevention features 880 may be coupled to inner shaft 890 via glue, welding or any suitable mechanisms to reinforce the weakest point of the inner shaft 890 adjacent trailing pillow 852b. In at least some examples, a single kink prevention feature is used. Alternatively, multiple kink prevention features may be used including forming kink prevention features adjacent each of the pillows. The kink prevention features may also extend the length of the balloon and/or into the PHV section of the balloon.

Any one or more of the features described herein (the terminal pillows, intermediate pillows, shoulders, overhanging pillows, kink resistance features, internal retention features, etc.) may be used, alone or in combination, to improve trackability, minimize transition along a delivery device length, and/or improve valve retention during delivery or deployment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. Further, it should be understood that different embodiments described herein may be combined with other embodiments described herein to achieve the benefits of both embodiments.

1. A delivery device, comprising:
a catheter; and
an inflatable balloon coupled to the catheter, the inflatable balloon forming a leading pillow and a trailing pillow spaced from the leading pillow, the leading pillow and the trailing pillow defining a valve seat therebetween to retain a prosthetic heart valve during tracking of the delivery device.

2. A system comprising:
the delivery device of claim 1; and
a prosthetic heart valve comprising a stent and a valve assembly.

3. The delivery device of claim 1, further comprising an intermediate pillow disposed between the leading pillow and the trailing pillow.

4. The delivery device of claim 3, wherein the intermediate pillow is shorter than at least one of the leading pillow and the trailing pillow in a radial direction.

5. The delivery device of claim 3, wherein the intermediate pillow is shorter than at least one of the leading pillow and the trailing pillow in an axial direction.

6. The delivery device of claim 3, wherein the intermediate pillow and the leading pillow define a valve cavity configured and arranged to accept a majority of a valve assembly.

7. The delivery device of claim 3, wherein the intermediate pillow is disposed equidistant to the leading pillow and the trailing pillow.

8. The delivery device of claim 3, wherein the intermediate pillow is disposed closer to the leading pillow than the trailing pillow.

9. The delivery device of claim 3, wherein the intermediate pillow is disposed closer to the trailing pillow than the leading pillow.

10. The system of claim 2, wherein the leading pillow and the trailing pillow extend radially outward farther than the prosthetic heart valve.

11. The system of claim 2, wherein the leading pillow and the trailing pillow overhang edges of the stent of the prosthetic heart valve.

12. The system of claim 2, further comprising a loader sheath having a radially inward protruding ramp to retain the stent close to the balloon during a deairing process.

13. The delivery device of claim 1, further comprising an inner shaft extending through the balloon and having at least one rigid kink feature disposed adjacent at least one of the leading pillow and the trailing pillow.

14. The delivery device of claim 13, wherein the at least one rigid kink feature is disposed adjacent the trailing pillow.

15. The delivery device of claim 1, further comprising an inner shaft having at least one cage disposed within the balloon.

16. The delivery device of claim 15, wherein the inner shaft comprises at least one of a helical tube and a braided tube.

17. A method of delivering a prosthetic heart valve, the method comprising:
providing a delivery device having a catheter and an inflatable balloon coupled to the catheter;
forming a leading pillow and a trailing pillow spaced from the leading pillow on the balloon, the leading pillow and the trailing pillow defining a valve seat;
placing a prosthetic heart valve on the valve seat; and
advancing the delivery device to a native aortic valve of a patient while the prosthetic heart valve is disposed between the leading pillow and the trailing pillow of the delivery device.

18. The method of claim 17, wherein forming a leading pillow and a trailing pillow comprises heat setting the balloon to form the leading pillow and the trailing pillow prior to placing the prosthetic heart valve on the valve seat.

19. The method of claim 17, further comprising forming an intermediate pillow between the leading pillow and the trailing pillow.

20. The method of claim 19, constraining a valve assembly of the prosthetic heart valve between the intermediate pillow and the leading pillow.
